(19) United States
(12) Patent Application Publication
(10) Pub. No.: US 2020/0312428 A1
(43) Pub. Date: Oct. 1, 2020

(54) SmartLabs Processor

(71) Applicant: GE Precision Healthcare LLC, Wauwatosa, WI (US)

(72) Inventors: Barbara Allison Reeves, Pittsburgh, PA (US); Christopher Deible, Pittsburgh, PA (US)

(21) Appl. No.: 16/369,587

(22) Filed: Mar. 29, 2019

(51) Int. Cl. G16H 10/40; G16H 10/60; A61B 5/00; G16H 80/00

(52) U.S. Cl. CPC G16H 10/40; G16H 10/60; G16H 15/00; G16H 80/00; A61B 5/742

(57) ABSTRACT

Methods, apparatus, systems and articles of manufacture are disclosed to generate an interface definition based on role, exam terms, and rules. An example apparatus includes at least one processor to at least: trigger rule generation based on an identified role and a primary exam; extract terms from the primary exam; process the extracted terms and identified role according to a first set of rules to generate a first lab panel display in an interface; facilitate i) rule editing and/or ii) rule generation to form a second set of rules using the extracted terms and identified role; apply the second set of rules to: i) modify the first lab panel display; and/or ii) generate a second lab panel display; generate an interface definition based on the first lab panel display and the second lab panel display; and provide the interface definition as output for display and interaction.

### FIELD OF THE DISCLOSURE

This disclosure relates generally to interface generation, and, more particularly, to systems and methods to process lab-related rules and exam terms to generate an interface.

### BACKGROUND

The statements in this section merely provide background information related to the disclosure and may not constitute prior art.

Healthcare environments, such as hospitals or clinics, include information systems, such as hospital information systems (HIS), radiology information systems (RIS), clinical information systems (CIS), and cardiovascular information systems (CVIS), and storage systems, such as picture archiving and communication systems (PACS), library information systems (LIS), and electronic medical records (EMR). Information stored can include patient medication orders, medical histories, imaging data, laboratory test results, diagnosis information, management information, and/or scheduling information, for example. A wealth of information is available, but the information can be siloed in various separate systems requiring separate access, search, and retrieval. Correlations between healthcare data remain elusive due to technological limitations on the associated systems.

Further, when data is brought together for display, the amount of data can be overwhelming and confusing. Such data overload presents difficulties when trying to display, and competing priorities put a premium in available screen real estate. Existing solutions are deficient in addressing these and other related concerns.

### BRIEF DESCRIPTION OF THE DRAWINGS

FIG. 1 is a schematic illustration of an example workflow manager.

FIG. 2 depicts an example graphical user interface to display output of the workflow manager of FIG. 1 and associated layers.

FIG. 3 illustrates an example implementation of a lab interface processor of the example workflow manager of FIG. 1

FIGS. 4A-4F show example interface panels to set preferences and create rules via the example interface of FIG. 2.

FIGS. 5-6 are flowcharts representative of machine readable instructions which may be executed to implement the apparatus of FIGS. 1-4.

FIGS. 7-8 are block diagrams of an example processing platform structured to execute the instructions of FIGS. 5-6 to implement the apparatus of FIGS. 1-4.

The figures are not to scale. Instead, the thickness, size, proportionality, etc., of the layers, regions, etc., may be enlarged in the drawings. In general, the same reference numbers will be used throughout the drawing(s) and accompanying written description to refer to the same or like parts. As used in this patent, stating that any part (e.g., a layer, area, region, portion, etc.) is in any way on (e.g., positioned on, located on, disposed on, or formed on, etc.) another part, indicates that the referenced part is either in contact with the other part, or that the referenced part is above the other part, adjacent to the part, etc., with one or more intermediate part(s) located therebetween. Stating that any part is in contact with another part means that there is no intermediate part between the two parts. Although the figures show layers and regions with clean lines and boundaries, some or all of these lines and/or boundaries may be idealized. In reality, the boundaries and/or lines may be unobservable, blended, and/or irregular.

Descriptors “first,” “second,” “third,” etc. are used herein when identifying multiple elements or components which may be referred to separately. Unless otherwise specified or understood based on their context of use, such descriptors are not intended to impute any meaning of priority or ordering in time but merely as labels for referring to multiple elements or components separately for ease of understanding the disclosed examples. In some examples, the descriptor “first” may be used to refer to an element in the detailed description, while the same element may be referred to in a claim with a different descriptor such as “second” or “third.” In such instances, it should be understood that such descriptors are used merely for ease of referencing multiple elements or components.

### BRIEF SUMMARY

Certain examples provide an apparatus including memory storing instructions and at least one processor. The at least one processor is to execute the instructions to at least: trigger rule generation based on an identified role and a primary exam; extract terms from a record of the primary exam; process the extracted terms and the identified role according to a first set of rules to generate a first lab panel display in an interface; facilitate at least one of i) rule editing or ii) rule generation to form a second set of rules using the extracted terms and the identified role; apply the second set of rules to at least one of: i) modify the first lab panel display; or ii) generate a second lab panel display; generate an interface definition based on the first lab panel display and the second lab panel display; and provide the interface definition as output for display and interaction.

Certain examples provide at least one tangible computer-readable storage medium including instructions that, when executed cause at least one processor to at least: trigger rule generation based on an identified role and a primary exam; extract terms from a record of the primary exam; process the extracted terms and the identified role according to a first set of rules to generate a first lab panel display in an interface; facilitate at least one of i) rule editing or ii) rule generation to form a second set of rules using the extracted terms and the identified role; apply the second set of rules to at least one of: i) modify the first lab panel display; or ii) generate a second lab panel display; generate an interface definition based on the first lab panel display and the second lab panel display; and provide the interface definition as output for display and interaction.

Certain examples provide a method including triggering, by executing an instruction using at least one processor, rule generation based on an identified role and a primary exam. The example method includes extracting, by executing an instruction using the at least one processor, terms from a record of the primary exam. The example method includes processing, by executing an instruction using the at least one processor, the extracted terms and the identified role according to a first set of rules to generate a first lab panel display in an interface. The example method includes facilitating, by executing an instruction using the at least one processor, at least one of i) rule editing or ii) rule generation to form a second set of rules using the extracted terms and the identified role. The example method includes applying, by executing an instruction using the at least one processor, the second set of rules to at least one of: i) modify the first lab panel display; or ii) generate a second lab panel display. The example method includes generating, by executing an instruction using the at least one processor, an interface definition based on the first lab panel display and the second lab panel display. The example method includes providing, by executing an instruction using the at least one processor, the interface definition as output for display and interaction.

### DETAILED DESCRIPTION

In the following detailed description, reference is made to the accompanying drawings that form a part hereof, and in which is shown by way of illustration specific examples that may be practiced. These examples are described in sufficient detail to enable one skilled in the art to practice the subject matter, and it is to be understood that other examples may be utilized and that logical, mechanical, electrical and other changes may be made without departing from the scope of the subject matter of this disclosure. The following detailed description is, therefore, provided to describe an exemplary implementation and not to be taken as limiting on the scope of the subject matter described in this disclosure. Certain features from different aspects of the following description may be combined to form yet new aspects of the subject matter discussed below.

When introducing elements of various embodiments of the present disclosure, the articles “a,” “an,” and “the” are intended to mean that there are one or more of the elements. The terms “first,” “second,” and the like, do not denote any order, quantity, or importance, but rather are used to distinguish one element from another. The terms “comprising,” “including,” and “having” are intended to be inclusive and mean that there may be additional elements other than the listed elements. As the terms “connected to,” “coupled to,” etc. are used herein, one object (e.g., a material, element, structure, member, etc.) can be connected to or coupled to another object regardless of whether the one object is directly connected or coupled to the other object or whether there are one or more intervening objects between the one object and the other object.

As used herein, the terms “system,” “unit,” “module,” “engine,” etc., may include a hardware and/or software system that operates to perform one or more functions. For example, a module, unit, or system may include a computer processor, controller, and/or other logic-based device that performs operations based on instructions stored on a tangible and non-transitory computer readable storage medium, such as a computer memory. Alternatively, a module, unit, engine, or system may include a hard-wired device that performs operations based on hard-wired logic of the device. Various modules, units, engines, and/or systems shown in the attached figures may represent the hardware that operates based on software or hardwired instructions, the software that directs hardware to perform the operations, or a combination thereof.

In addition, it should be understood that references to “one embodiment” or “an embodiment” of the present disclosure are not intended to be interpreted as excluding the existence of additional embodiments that also incorporate the recited features.

Aspects disclosed and described herein provide systems and associated methods to view laboratory test and result information such as labs relevant (e.g., most relevant, etc.) to a primary exam being read. Thus, certain examples provide a tool for radiologists (e.g., a SmartLabs tool, etc.) that reduces information overload by only showing lab(s) that are most relevant to a primary exam being read.

For example, the tool can initially run with a default short list of labs to be displayed based on a user's radiology specialty. As a user selects to display additional panel types, the tool prompts users to create one or more “rules” that use key terms to trigger specific panel types/order sets to be displayed on a case-by-case basis.

No prior tools are available attempt to intelligently record and present a redacted view of labs information to users such as radiologists or users focused on the interpretation of medical imaging. Today, users encounter labs information in the context of an electronic medical record (EMR) in which all labs information is presented in a default view where all labs are visible, with an ability to manually filter by type. In contrast, certain examples provide an interface and task-flows associated with creating views of labs information that are unique including recording and thereafter presenting a user's preferences, etc.

Users such as radiologists, other healthcare practitioners involved in interpretation of medical images, etc., can benefit from such systems and methods as described herein. Certain examples help to automate interpretation of medical images to create a diagnostic report of image findings and/or other conclusions based on medical imaging, etc.

In certain examples, when a user such as a radiologist, etc., uses the tool for the first time, the user identifies a subspecialty (e.g., abdominal imaging, breast imaging, cardiac imaging, emergency radiology, musculoskeletal imaging, neurointerventional radiology, neuroradiology, nuclear medicine, pediatric radiology, thoracic imaging, vascular interventional radiology, etc.), which allows the system to display a default subset of labs that are most common to the particular subspecialty via a graphical user interface associated with the tool. Based on the first-time display, the subset of labs can become a default view based on a radiologist's and/or other user's specialty, for example. When a user selects a new panel type to be displayed, the tool is updated to include the new panel in the display.

In certain examples, one or more rules can be provided, specified by the system based on user role/type and/or available information, input by the user, etc. For example, the tool can create a rule to display a specific panel and/or order set based on terms found in the primary exam information. Mention(s) of key term(s) (e.g., anatomy, condition, patient name, etc.) are classified with an identifying tag and act as triggers to automatically display this panel/order set in the future.

The terms can form a criteria list or included terms list for filtering, ordering, highlighting, reporting, etc. If a user removes a term from the criteria list, the term is still available below the included terms list. A user can add terms back to the included terms list. If no terms are removed, this element is absent, for example. The tool “remembers” which term(s) are excluded as triggers for a panel type and does not ask about adding that term in the same future, for example.

In certain examples, the user is prompted to add term(s) each time new term(s) is/are present and a panel is added. Over time, a user may see prompts less often as conditions and anatomy are added or excluded from the user's rule set.

The user can close the tool and/or use the tool to create a new rule, for example. If the user indicates that a rule creation dialog should not be shown again via the user interface, the tools uses defaults for the user's specialty and user manual input, if provided, to automatically create rule(s), for example.

In certain examples, the tool is accessible from within a lab's category settings, from a user's preferences/settings, etc. The “SmartLabs” tool can be turned on or off from a preferences sections of a laboratory review application, an image reader, etc.

Panels and associated rules can be established for a variety of healthcare areas such as immunology, urinalysis, common chemistry, blood bank, Chem 7, chemistry, endocrinology, hematology, microbiology, special chemistry, thyroid, urine culture, etc. Each panel type has a rule set that can be edited. A limited number of criteria is/are displayed when list items are collapsed, but users can expand a list item to see all criteria. The date a rule set was last updated can also be displayed.

When a rule set is in edit mode, terms can be added and removed. Changes are automatically saved, and a time stamp updates with each change. A user can leave the edit state by: clicking and/or otherwise selecting anywhere outside the rule set being edited, clicking/selecting edit again, closing the smart labs panel in the graphical user interface, etc.

In certain examples, a user can add new terms. Example types of terms include: anatomy, condition, demographics, severity (e.g., critical, acute, etc.), etc. Rules are either created by the “user” or they are “system generated”. System-generated rules are not editable, and lack an “edit” functionality, for example.

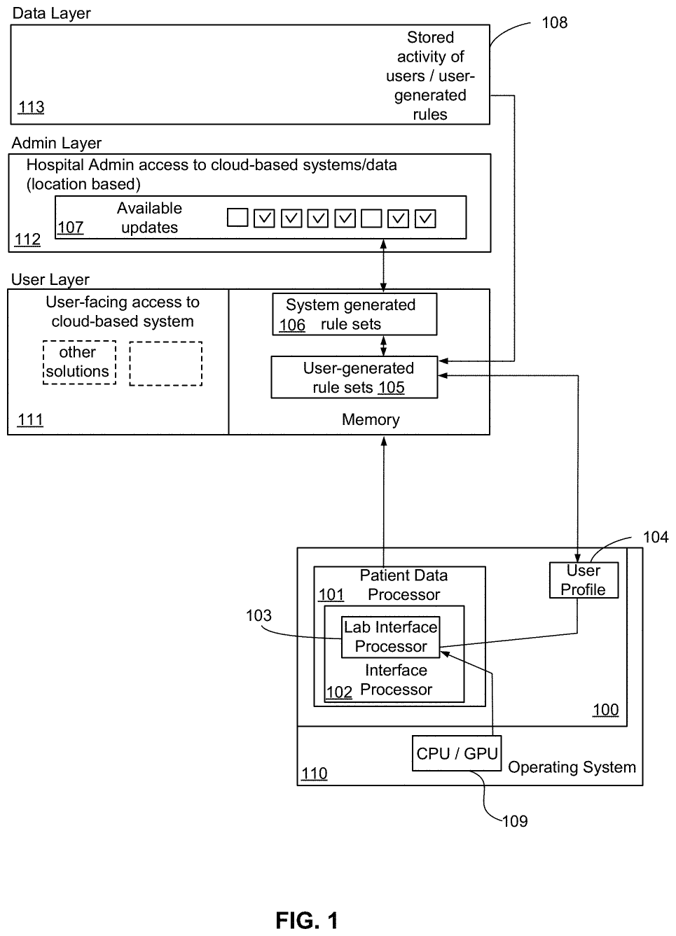

FIG. 1 illustrates an example workflow manager 100 including “smart” healthcare solution system. Using the system 100, rule set(s) can be created, updated, modified, etc. Certain examples enable creation, modification, deployment, etc., with reduced (e.g., minimal, etc.) disruption to user workflow and improved (e.g., maximum, etc.) opportunity to capture data to improve diagnosis, treatment, and/or other user experience, etc. Certain examples include a user layer 111, an administration layer 112, and a data layer 113, in which the data layer 113 provides oversight to moderate and improve performance of automated rule generation, term filtration, graphical user interface configuration, etc., over time based on monitored data, feedback, etc.

FIG. 1 is a schematic illustration of the workflow manager 100 including a patient data processor 101, an interface processor 102, and a lab interface processor 103. A user profile 104 provides information to the lab interface processor 103 and accepts feed back such as a user-generated rule set 105. The workflow manager 100 is driven by a processor 109 such as a central processing unit (CPU), general processing unit (GPU), etc., and executes with respect to an operating system 110, for example. Thus, the workflow manager 100 alters behavior of the operating system 110 and modifies the CPU/GPU 109 to drive the healthcare solution system.

As shown in the example of FIG. 1, drives processing, display, and interaction with respect to patient data including images, image related clinical content (IRCC), clinical history, etc. The patient data processor 101 implements a user layer 111, an administration layer 112, and a data layer 113. The user layer 111 is operated by individual user(s) and provides user-facing access to the “smart” solutions provided by the workflow manager 100, which can be implemented locally, as a cloud-based system, provided via an edge device, etc. The user layer 111 can host rule sets 105, 106 such as user-generated rule set(s) 105, a system-generated rule set(s) 106, etc. The user-generated rule set 105 can be provided to update the user profile 104, for example. The administration or admin layer 112 is operated by administrator(s) such as hospital administrators, hospital systems, other location administrator personnel/devices, etc. The data layer 113 is operated by data scientists, analysts, and associated systems, for example.

In the example of FIG. 1, the workflow manager 100 can be implemented as an installed computer application, and the interface processor 102 is a cloud-based component that involves web browser and/or other Internet-based connectivity. The interface processor 102 and its lab interface processor 103 can be triggered as part of the patient data processor 101 when the workflow manager 100 is launched, for example.

In certain examples, the patient data processor 101, the interface processor 102, and the lab interface processor 103 are implemented separately. In certain examples, the patient data processor 101, the interface processor 102, and the lab interface processor 103 are implemented together. In certain examples, the patient data processor 101, the interface processor 102, and the lab interface processor 103 are implemented in hardware circuitry. In certain examples, the patient data processor 101, the interface processor 102, and the lab interface processor 103 are implemented as a combination of hardware and firmware. In certain examples, the patient data processor 101, the interface processor 102, and the lab interface processor 103 are implemented as a combination of hardware, firmware, and/or software. In certain examples, the patient data processor 101, the interface processor 102, and the lab interface processor 103 are implemented as one or more virtual machines, containers, and/or other computer processing constructs.

In certain examples, the user profile 104 is associated with a cloud-based user account that stores user-generated rule sets 105. Using the cloud-based profile 104, rules can be synchronized between the cloud and one more local workstations, other systems, etc. In the user layer 111, the local system pulls updates from the administration layer 112 when a user logs in and pushes updates to the data layer 113 when the user logs out, for example. Thus, the user's activity is available for analysis, and updates/improvements pushed by the administration 112 and data 113 layers can be made available to the user of the workflow manager 100, for example.

In certain examples, system-generated rule set(s) 106 are intended to compliment user-generated rule sets 105. User-generated rule sets 105 can override system-generated rule sets 106 if the system-generated rule set(s) 106 are marked (e.g., by the admin layer 112, etc.) as non-mandatory. In certain examples, users and administrators are associated with a specific hospital system and location.

In certain examples, a user-generated rule set 105 is a selection of labs and/or other healthcare data to be displayed as specified by a user. The user-generated rule set 105 can provide patient health data (e.g., lab results, etc.) display based on specified characteristic(s) of a primary exam for the patient, for example.

In certain examples, a system-generated rule set 106 is a default selection of labs and/or other healthcare data to be displayed. The system-generated rule set 106 can be made available automatically based on characteristic(s) of the primary exam by the smart solutions system 100. System-generated rules can be mandatory or optional to implement by local hospital and/or other health system administrator. For example, a mandatory rule can include a rule associated with an FDA regulation, etc. An optional rule can include a rule tailored to a small hospital, radiology subspecialty, etc., that may not be added by all systems, for example.

In certain examples, hospital and/or other health system (e.g., location-related, etc.) administrators can approve/activate updates 107 via the admin layer 112. Administrators can view available updates 107, whether active or inactive, and trigger application of one or more updates 107 to the system-generate rule set(s) 106, for example.

At the data layer 113, user activity and user-generated rule sets 105 are stored 108 and analyzed for patterns that can be used to generate common rules that can be added as a standard to system-generated rule sets 106. User-generated rule set(s) 105 can also be stored on local computer/workstation memory 109 until a user logs out, for example. Application memory and user-generated rules storage is compatible with the operating system 110, for example.

Using the workflow manager 100 and its layers 111-113, lab panels and/or other order sets representing a group of results (e.g., lab test results, etc.) that have been ordered by a care provider, for example, can be processed, displayed, stored, routed, etc. Some panels, such as “Common chem”, etc., are a standard 8-14 group of tests common across hospital systems. Hospitals and lab providers can also generate new panels or “order sets” of tests, for example. The example lab interface processor 103 can recognizes groupings of results and display them in a panel driver, for example.

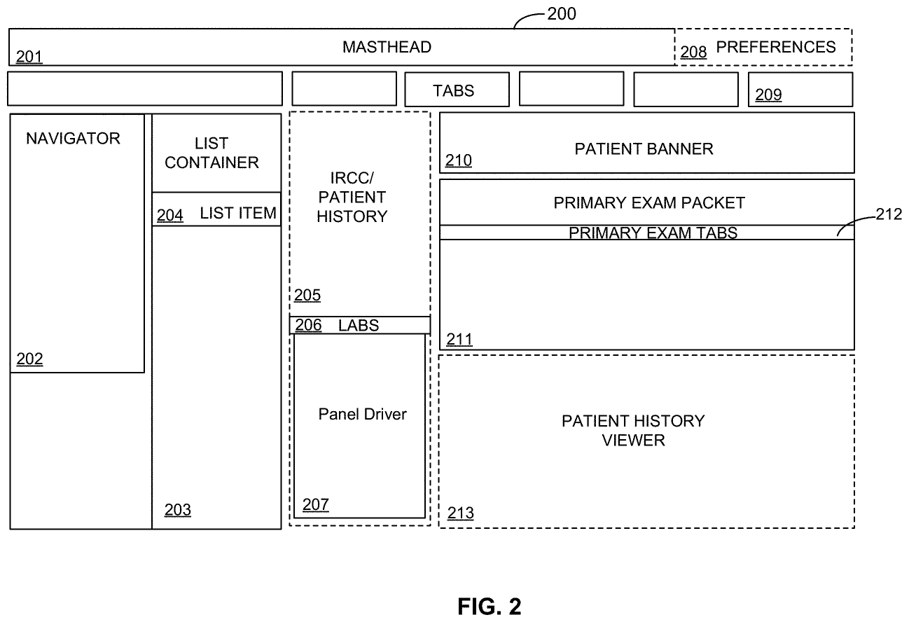

FIG. 2 depicts an example graphical user interface 200 to display output of the workflow manager 100 and associated layers 111-113. As shown in the example of FIG. 2, a masthead 201 can identify a patient, exam, other context, etc. A navigator 202 provides one or more options to display patient exam data, history, etc., for a selected patient. The list container 203 includes one or more list items 204 including worklist items, patient records, exams, etc.

Image related clinical content (IRCC) 205 includes patient history and/or other patient health data related to image content displayed in an exam for the patient. IRCC information 205 is provided in conjunction with lab content 206 and a panel driver 207. The IRCC 205 can provide clinical history, lab content 206, etc., to enable a more accurate interpretation of images and improve patient outcomes through higher quality care, for example. The panel driver 207 provides lab panel(s), order list(s), etc., to be displayed when an exam is loaded, for example. The panel driver 207 displays output from the lab interface processor 103 of the workflow manager 100.

The example interface 200 further provides information regarding preferences 208 and an ability to set the preferences 208. One or more tabs 209 provide access to opened exams. The patient banner 210 provides certain summary, demographic, and/or other identifying information for a patient associated with a selected exam. The primary exam packet 211 displays one or more exams for the patient via one or more primary exam tabs 212. A patient history viewer 213 provides a more detailed view of patient history selected from the IRCC module 205.

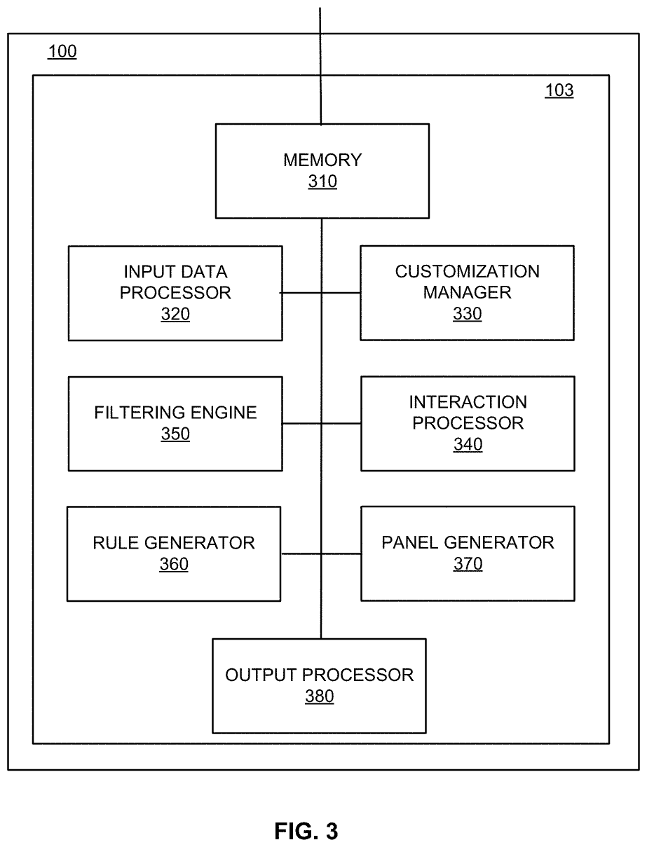

FIG. 3 illustrates an example implementation of the lab interface processor 103 of the workflow manager 100. The example processor 103 includes memory 310, an input data processor 320, a customization manager 330, an interaction processor 340, a filtering engine 350, a rule generator 360, a panel generator 370, and an output processor 380.

In the example of FIG. 3, the lab interface processor 103 receives input, such as an exam selection, worklist entry activation, clinical history access, clinical record update, user configuration, etc. The input is stored in memory 310 as one or more data structures that alter the memory 310 and transform the memory 310 into the particular data structure(s) representing the input. The input stored in memory 310 is also processed by the input data processor 320. The input data processor 320 processes the input to transform the input for use by the customization manager 330 and other elements of the processor 103. For example, the input data processor 320 can extract terms from an exam to be reviewed, patient order, patient medical record, etc. The input data processor 320 can extract exam parameters, instructions, other context information, relevancy factors, etc., to be used by the customization manager 300 to generate and/or otherwise customize one or more rules, graphical user interfaces, settings, etc., in the workflow manager 300.

The customization manager 330 processes the input (e.g., extracted terms, etc.) to transform the input into the interface 200 including IRCC/patient history 205, labs 206, panel driver 207, etc. The customization manager 330 works with the interaction processor 340 to drive the user layer 111, admin layer 112, and data layer 113 to provide labs and other information to the user interface display 200 based on system rules 106, user rules 105, updates 107, activity, extracted terms, etc. The interaction processor 340 processes user activity, system activity, user/system input, etc., to drive rules, updates, etc., in conjunction with the customization manager 330 to produce an updated interface display 200.

In certain examples, the customization manager 330 is triggered based on initiation of a process such as launching the workflow manager 100, selecting an item from the worklist 203, selection/specification of a user role (e.g., radiologist, cardiologist, neurologist, imaging technician, nurse, administrator, other clinician, etc.). Identification of context (e.g., patient context, exam context, user context, etc.) can automatically set parameters/settings for customization of the interface 200, for example. The context can specify or help to specify: patient context such as an identification of a patient and associated condition(s), data ownership, other patient characterizing information, etc.; exam context such as patient subject to examination, reason for examination, other information characterizing the exam, etc.; user context such as user settings/preferences, user profile, user location, user resources, etc.; etc.

In certain examples, the filtering engine 350 can be leveraged by the customization manager 330 to filter extracted terms and/or other information based on context (e.g., patient context, exam context, and/or user context, etc.), priority (e.g., urgent, high, medium, low, etc.), reason for exam, anatomy, condition, demographics, etc. Example terms can include anatomy, condition, demographics, severity (e.g., critical, acute, etc.), etc. The rule generator 360 can process customization information from the customization manager 350, interaction information from the interaction processor 340, filtered results from the filtering engine 350, etc., to generate one or more rules driving display of lab panel and other patient/exam information.

In certain examples, the panel generator 370 generates one or more panels (e.g., a lab panel 206, other panel driver 207, etc., forming part of the user interface display 200 based on the rule(s) from the rule generator 360, filtered results from the filtering engine 350, customization information from the customization manager 350, interaction information from the interaction processor 340, etc. The output processor 380 generates the user interface 200 from the output of the customization manager 330, interaction processor 340, filtering engine 350, rule generator 360, panel generator 370, etc. The output processor 380 can generate the user interface display 200 for interaction, route information for storage and/or further processing, trigger activation of an external device/system based on output, etc.

In certain examples, the interaction processor 340 can facilitate rule creation, rule editing/adjustment, etc., by taking user input and/or system feedback and providing input to the rule generator 360 to create a new panel display rule, edit an existing rule, etc. In certain examples, the interaction processor 340 can process user and/or other feedback input received by the input data processor 320 to drive the customization manager 330 to trigger the rule generator 360 to create a rule, edit a rule, etc., to provide the panel generator 370 with guidelines/requirements for the output processor 380 to generate and output the user interface 200 definition.

Thus, the example lab interface processor 103 drives processing, customization, display, and interaction with all or part of the interface 200, for example. For example, the example processor 103 can drive a diagnostic hub with documents and an initial display of information with relevant clinical documents, surgical notes, etc., based on an open exam, patient history, etc. As a user, program, and/or system navigates an examination and its result, the processor 103 can learn from the patterns, outcomes, and positive and negative feedback to improve display and interaction through the graphical user interface 200 in a “smart” process, for example.

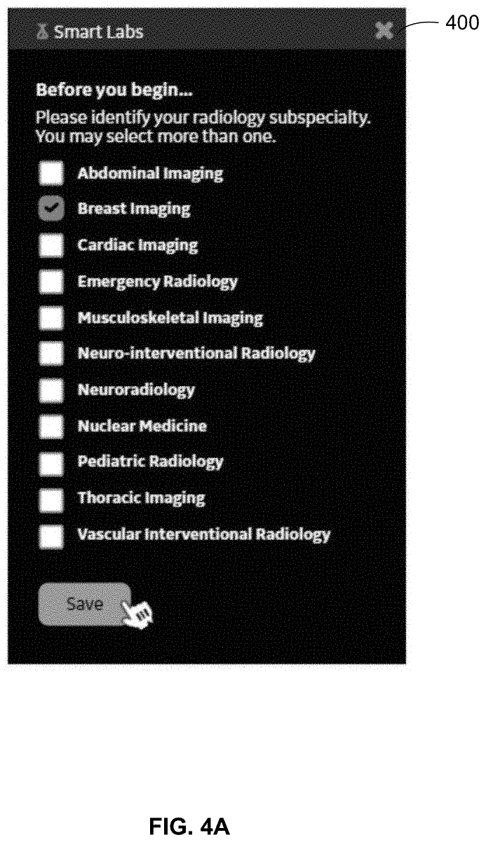

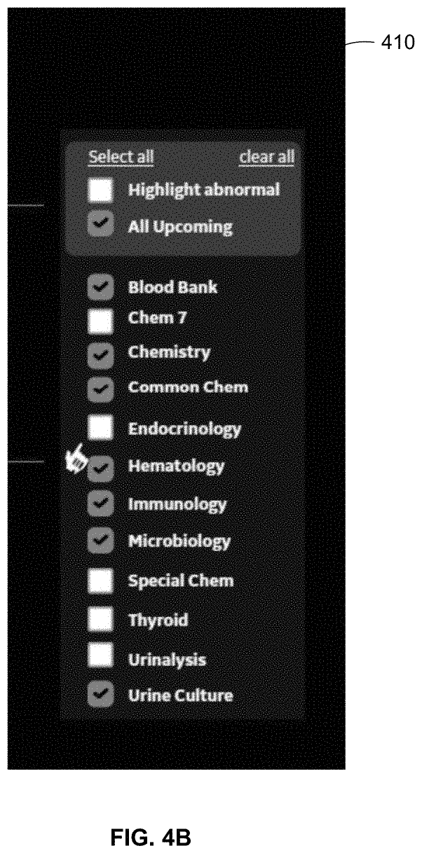

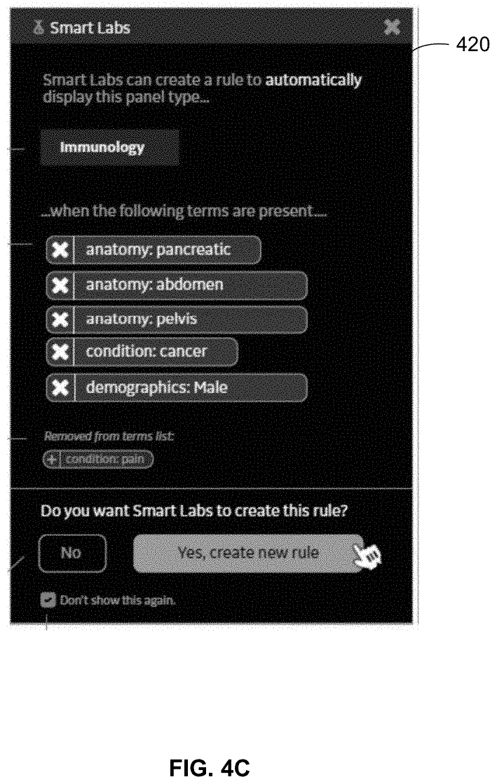

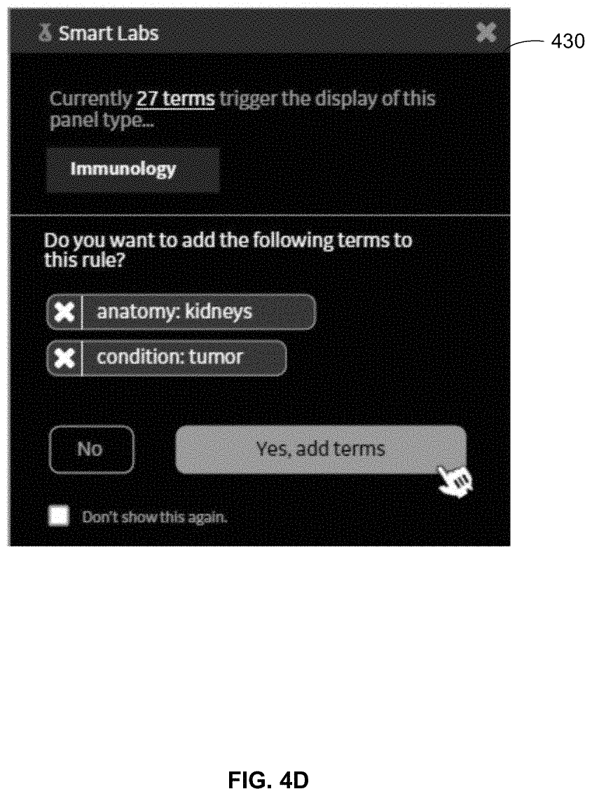

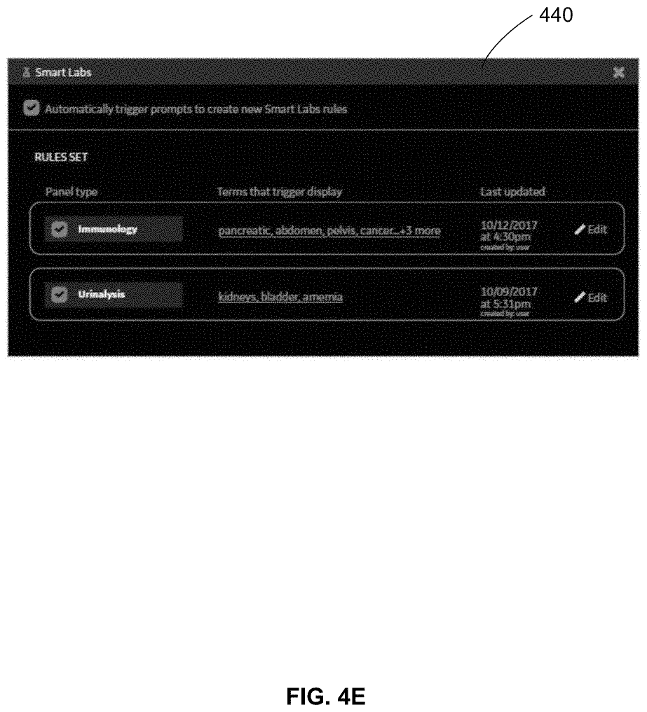

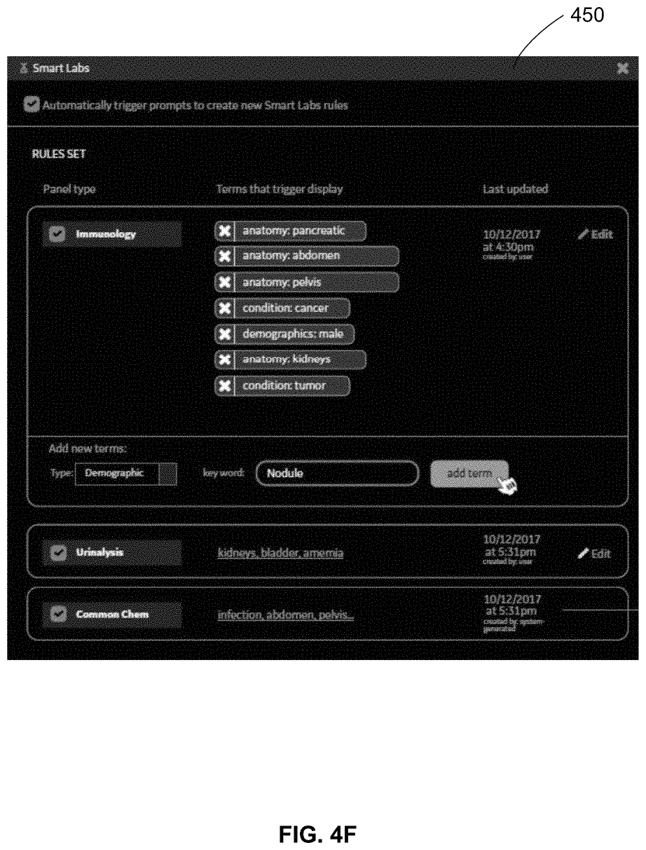

In certain examples, subspecialty lab panel preferences are a starting point for a default state showing relevant panel(s) driven by the processor 103 in the interface 200. When a user adds or subtracts a panel type from the default set, the lab interface processor 103 triggers rule creation. The lab interface processor 103 facilitates creation of one or more rule sets for automatic lab panel display. In certain examples, the lab interface processor 103 includes a text filter to extract term(s) from a primary exam's data. Terms include modality, body part, reason for exam (RFE), etc. Example interface panels displayed via the interface 200 are shown in FIGS. 4A-4F.

In certain examples, a first time use triggers display of a subspecialty interface panel 400 (see, e.g., FIG. 4A) to allow a user to identify their subspecialty(-ies). Identification of subspecialty allows the lab interface processor 103 to transform the interface panel to display a default subset 410 of labs (see, e.g., FIG. 4B) that are most common to associated subspecialty(-ies). The default display 410 shows a subset of labs/results for selection. Selected panel types are displayed, and unselected panel types are not displayed, for example. Selection of a panel type that was not selected by default triggers the lab interface processor 103 to create a rule associated with the selected panel type.

The lab interface processor 103 triggers a rule creation window 420, such as shown in the example of FIG. 4C, to enable creation of a rule to automatically display a panel/order set based on terms found in the primary exam information. The lab interface processor 103 creates a rule to display the selected panel based on terms found in the primary exam, for example. Mentions of “key” terms (e.g., anatomy, condition, patient name, etc.) are classified with an identifying tag and act as triggers to automatically display the corresponding panel type in future sessions, for example. If a user removes a term from the criteria list, the term is still available below the included terms or criteria list, as shown in the example of FIG. 4C. If no terms are removed, then this element is absent from the graphical user interface. The lab interface processor 103 remembers (e.g., stores in a data structure, etc.) which term(s) are excluded as triggers for a panel type and does not ask/prompt about adding those term(s) again in the future. Selecting “no” closes the panel 420, and selecting “yes” creates a new user-defined rule 105.

If a user selects “don't show this again” via the interface 420, then the lab interface processor 103 uses a default for the selected subspecialty along with any user manual inputs to display labs, for example. In certain examples, an additional option at a settings or system level includes deactivating the lab interface processor 103 if such assistance is undesired.

In certain examples, within the rule creation panel 420, terms are identified and classified within a category. Content can be structured such as using a tool and associated method to structure data prior to use using a content filter, algorithm, etc., to identify terms within a category such as condition—cancer, anatomy—abdomen, etc.

The lab interface processor 103 triggers a prompt or pop-up window 430, etc., to inform the user regarding terms, ask the user to clarify, suggest rule(s) to be added by the user, accept user input for rule creation, etc. For example, FIG. 4D shows a default panel set window 430 including a set of labs to be selected for inclusion or deselected for exclusion from a displayed panel set.

A frequency of such prompts decreases over time as the processor 103 learns user behavior/preferences, fleshes out the combined rule set of system-generated 106 and user-generated 105 rules, etc. For example, a user can select to not show the interface 430 again. However, if this box is not checked, the user sees a prompt to add terms each time when new terms are present and a panel is added. Over time, the user sees prompts less often as conditions and anatomy are added and/or excluded from the user's rule set 105. In certain examples, the user-generated rule set 105 can be saved and shared with another system to improve configuration of useful rule sets for particular sub-specialties, departmental systems, etc.

In certain examples, lab interface processor 103 settings can be edited within a labs category and/or within a user's global preferences. FIG. 4E shows an example rule set interface 440 that displays rule sets associated with panel types. A limited number of criteria are displayed when list items are collapsed, by a list item can be expanded to see all associated criteria. The date the rule set was last updated is also displayed.

The example expanded rule set interface 450 of FIG. 4F shows the full set of criteria for the associated rule. When a rule set is in edit mode, terms can be added and/or removed. Changes are automatically saved, and time stamps are associated with each update. The edit state can be exited by one or more of selecting outside the rule set being edited, selecting “edit” again, closing the panel 450, etc. Via the edit panel 450, new terms can be added such as anatomy, condition, demographics, severity (e.g., critical, acute, etc.), etc. Rules can be created by a user, system-generated, etc. In certain examples, system-generated rules are not editable and lack an “edit” option when reviewed in the panel 450. Thus, via the expanded interface 450, rule sets can be inspected and edited/added/removed, etc. Terms can be added independent of those terms pulled automatically from the primary exam.

In certain examples, a problem list can be used as a data source to trigger rule panel display. Key terms can be pulled from the problem list, for example.

Thus, certain examples provide a flexible interface driven by the lab interface processor 103 to accommodate a variety of user interaction. Some users can use a department's template set of rules and system defaults to “automatically” set up a majority of the rules they want applied. Some users may choose to add rules one by one until rule creation frequency diminishes. Some users may go to the “settings” section and modify the generic defaults, manually adjusting rules to match their preferences. Taking this approach, users can manually add terms to trigger panel launch, for example.

While an example implementation of the workflow manager 100 and its components is illustrated in FIGS. 1-4F, one or more of the elements, processes and/or devices illustrated in FIGS. 1-4F may be combined, divided, re-arranged, omitted, eliminated and/or implemented in any other way. Further, the example patient data processor 101, the example interface processor 102, the example lab interface processor 103, the example CPU/GPU 109, the example operating system 110, the example user layer 111, the example data layer 112, the example admin layer 113, and/or, more generally, the example workflow manager 100 of FIGS. 1-4F can be implemented by hardware, software, firmware and/or any combination of hardware, software and/or firmware. Thus, for example, any of the example patient data processor 101, the example interface processor 102, the example lab interface processor 103, the example CPU/GPU 109, the example operating system 110, the example user layer 111, the example data layer 112, the example admin layer 113, and/or, more generally, the example workflow manager 100 can be implemented by one or more analog or digital circuit(s), logic circuits, programmable processor(s), programmable controller(s), graphics processing unit(s) (GPU(s)), digital signal processor(s) (DSP(s)), application specific integrated circuit(s) (ASIC(s)), programmable logic device(s) (PLD(s)) and/or field programmable logic device(s) (FPLD(s)). When reading any of the apparatus or system claims of this patent to cover a purely software and/or firmware implementation, at least one of the example patient data processor 101, the example interface processor 102, the example lab interface processor 103, the example CPU/GPU 109, the example operating system 110, the example user layer 111, the example data layer 112, the example admin layer 113, is/are hereby expressly defined to include a non-transitory computer readable storage device or storage disk such as a memory, a digital versatile disk (DVD), a compact disk (CD), a Blu-ray disk, etc. including the software and/or firmware. Further still, the example workflow manager 100 may include one or more elements, processes and/or devices in addition to, or instead of, those illustrated in FIGS. 1-4F, and/or may include more than one of any or all of the illustrated elements, processes and devices. As used herein, the phrase “in communication,” including variations thereof, encompasses direct communication and/or indirect communication through one or more intermediary components, and does not require direct physical (e.g., wired) communication and/or constant communication, but rather additionally includes selective communication at periodic intervals, scheduled intervals, aperiodic intervals, and/or one-time events.

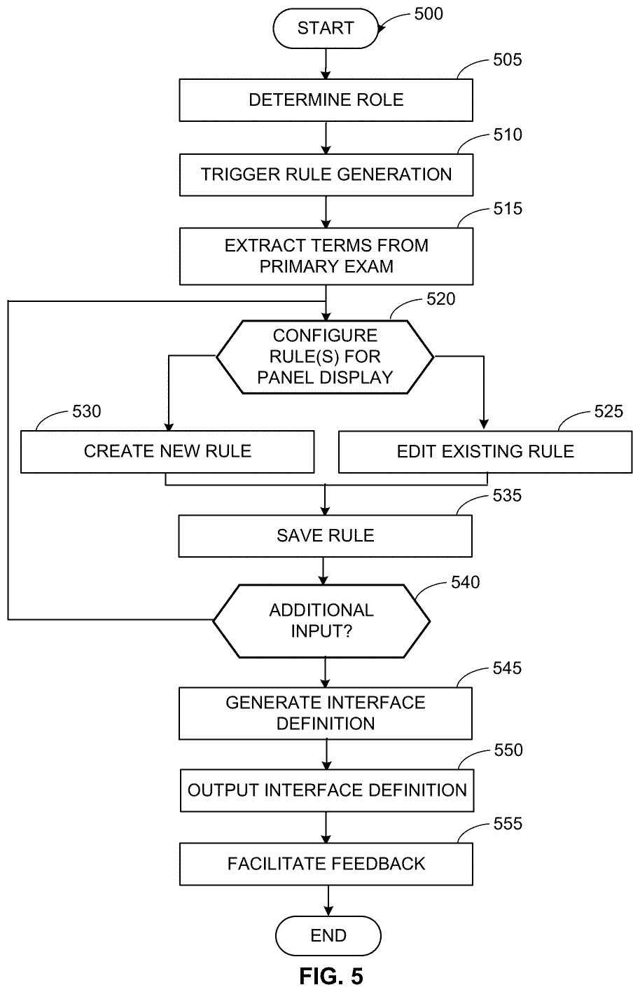

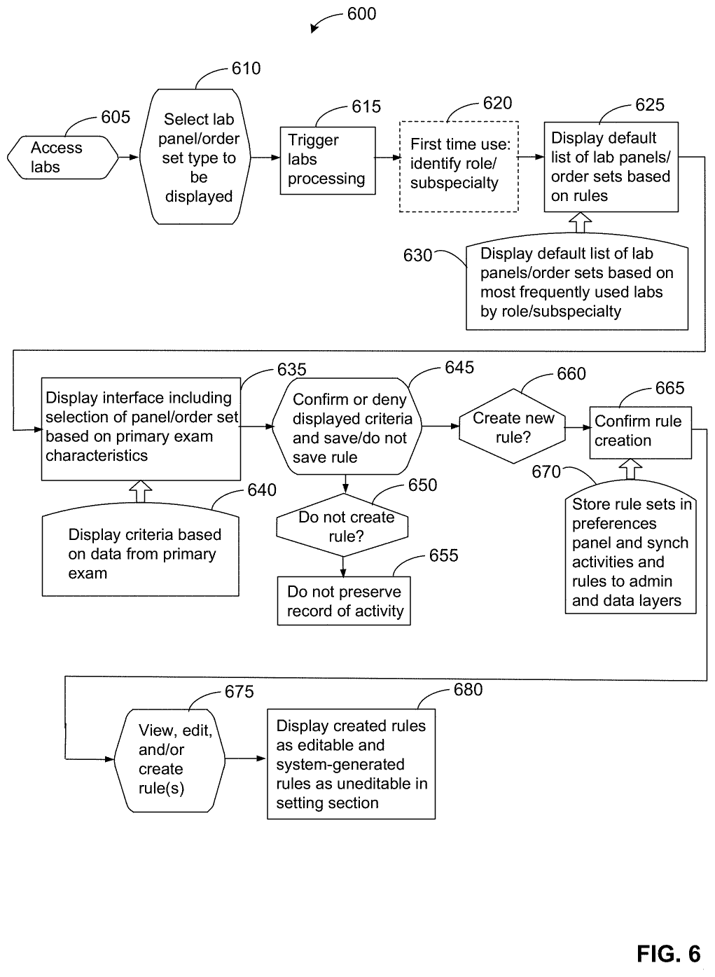

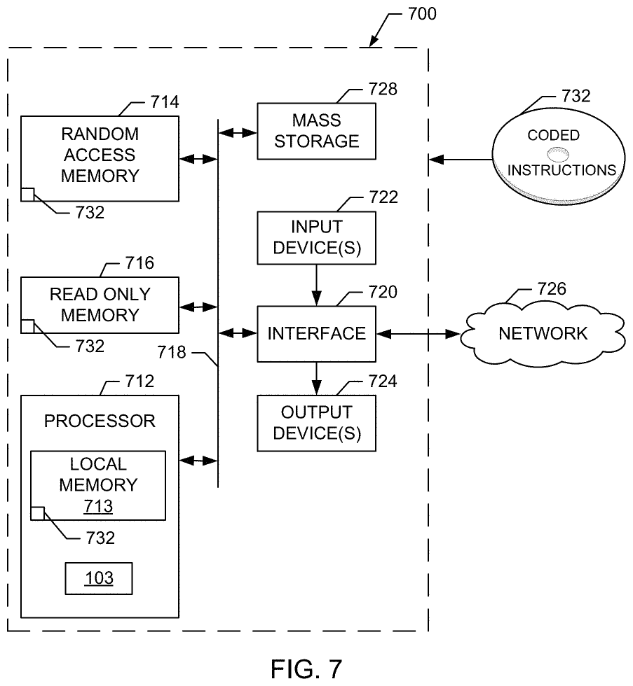

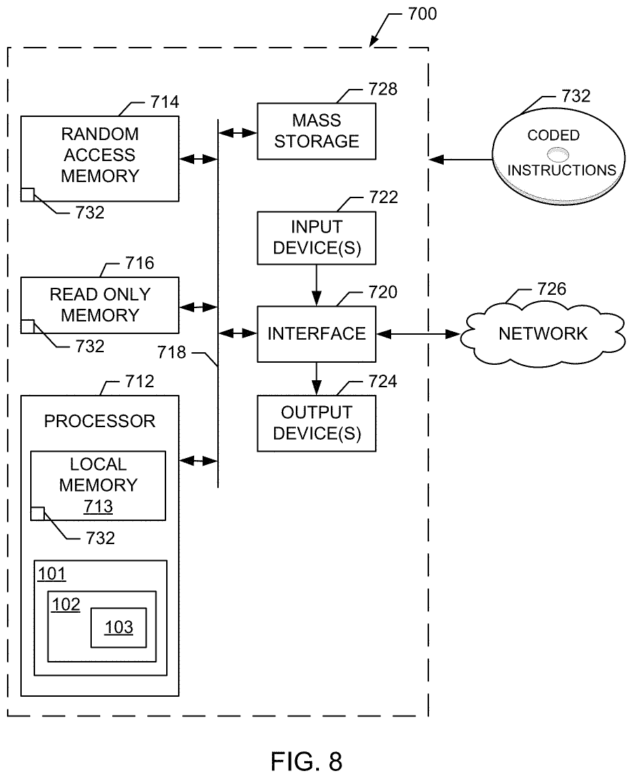

Flowcharts representative of example hardware logic, machine readable instructions, hardware implemented state machines, and/or any combination thereof for implementing the apparatus 100 of FIGS. 1-4F are shown in FIGS. 5-6. The machine readable instructions may be one or more executable programs or portion(s) of an executable program for execution by a computer processor such as the processor 712 shown in the example processor platform 700 discussed below in connection with FIGS. 7-8. The program may be embodied in software stored on a non-transitory computer readable storage medium such as a CD-ROM, a floppy disk, a hard drive, a DVD, a Blu-ray disk, or a memory associated with the processor 712, but the entire program and/or parts thereof could alternatively be executed by a device other than the processor 712 and/or embodied in firmware or dedicated hardware. Further, although the example program is described with reference to the flowcharts illustrated in FIGS. 5-6, many other methods of implementing the example apparatus 100 may alternatively be used. For example, the order of execution of the blocks may be changed, and/or some of the blocks described may be changed, eliminated, or combined. Additionally or alternatively, any or all of the blocks may be implemented by one or more hardware circuits (e.g., discrete and/or integrated analog and/or digital circuitry, an FPGA, an ASIC, a comparator, an operational-amplifier (op-amp), a logic circuit, etc.) structured to perform the corresponding operation without executing software or firmware.

The machine readable instructions described herein may be stored in one or more of a compressed format, an encrypted format, a fragmented format, a packaged format, etc. Machine readable instructions as described herein may be stored as data (e.g., portions of instructions, code, representations of code, etc.) that may be utilized to create, manufacture, and/or produce machine executable instructions. For example, the machine readable instructions may be fragmented and stored on one or more storage devices and/or computing devices (e.g., servers). The machine readable instructions may require one or more of installation, modification, adaptation, updating, combining, supplementing, configuring, decryption, decompression, unpacking, distribution, reassignment, etc. in order to make them directly readable and/or executable by a computing device and/or other machine. For example, the machine readable instructions may be stored in multiple parts, which are individually compressed, encrypted, and stored on separate computing devices, wherein the parts when decrypted, decompressed, and combined form a set of executable instructions that implement a program such as that described herein. In another example, the machine readable instructions may be stored in a state in which they may be read by a computer, but require addition of a library (e.g., a dynamic link library (DLL)), a software development kit (SDK), an application programming interface (API), etc. in order to execute the instructions on a particular computing device or other device. In another example, the machine readable instructions may need to be configured (e.g., settings stored, data input, network addresses recorded, etc.) before the machine readable instructions and/or the corresponding program(s) can be executed in whole or in part. Thus, the disclosed machine readable instructions and/or corresponding program(s) are intended to encompass such machine readable instructions and/or program(s) regardless of the particular format or state of the machine readable instructions and/or program(s) when stored or otherwise at rest or in transit.

As mentioned above, the example processes of FIGS. 5-6 may be implemented using executable instructions (e.g., computer and/or machine readable instructions) stored on a non-transitory computer and/or machine readable medium such as a hard disk drive, a flash memory, a read-only memory, a compact disk, a digital versatile disk, a cache, a random-access memory and/or any other storage device or storage disk in which information is stored for any duration (e.g., for extended time periods, permanently, for brief instances, for temporarily buffering, and/or for caching of the information). As used herein, the term non-transitory computer readable medium is expressly defined to include any type of computer readable storage device and/or storage disk and to exclude propagating signals and to exclude transmission media.

“Including” and “comprising” (and all forms and tenses thereof) are used herein to be open ended terms. Thus, whenever a claim employs any form of “include” or “comprise” (e.g., comprises, includes, comprising, including, having, etc.) as a preamble or within a claim recitation of any kind, it is to be understood that additional elements, terms, etc. may be present without falling outside the scope of the corresponding claim or recitation. As used herein, when the phrase “at least” is used as the transition term in, for example, a preamble of a claim, it is open-ended in the same manner as the term “comprising” and “including” are open ended. The term “and/or” when used, for example, in a form such as A, B, and/or C refers to any combination or subset of A, B, C such as (1) A alone, (2) B alone, (3) C alone, (4) A with B, (5) A with C, (6) B with C, and (7) A with B and with C. As used herein in the context of describing structures, components, items, objects and/or things, the phrase “at least one of A and B” is intended to refer to implementations including any of (1) at least one A, (2) at least one B, and (3) at least one A and at least one B. Similarly, as used herein in the context of describing structures, components, items, objects and/or things, the phrase “at least one of A or B” is intended to refer to implementations including any of (1) at least one A, (2) at least one B, and (3) at least one A and at least one B. As used herein in the context of describing the performance or execution of processes, instructions, actions, activities and/or steps, the phrase “at least one of A and B” is intended to refer to implementations including any of (1) at least one A, (2) at least one B, and (3) at least one A and at least one B. Similarly, as used herein in the context of describing the performance or execution of processes, instructions, actions, activities and/or steps, the phrase “at least one of A or B” is intended to refer to implementations including any of (1) at least one A, (2) at least one B, and (3) at least one A and at least one B.

The program 500 of FIG. 5 includes block 505, at which a role is selected. For example, a role can be selected for the workflow manager 100. A role can be selected for the user interface 200 driven by the workflow manager 100, for example. A role can be selected for a user of the interface 200, for example. The role can be selected based on and/or in conjunction with an exam (e.g., one or more imaging studies, associated lab results, other patient data, etc.) to be reviewed and/or otherwise processed, for example.

At block 510, rule generation is triggered. For example, based on the selected role and/or information regarding the exam, rule generation can be triggered to adjust the user-generated rule set 105 and/or the system-generated rule set 106.

At block 515, terms are extracted from the exam (e.g., the primary exam, the primary exam and one or more secondary exams, etc.) to be reviewed via the interface 200. For example, the exam record includes terms in a DICOM header, HL7 message, image annotation, patient electronic medical record data, patient identifier, reason for exam, other context information such as user, location, date, time, anatomy, region of interest, exam finding, etc. Example terms can include anatomy, condition, demographics, severity (e.g., critical, acute, etc.), etc. Extracted terms, taken from the exam via the filtering engine 350, for example, can be stored in a data store, in memory 310, as a construct for combination into a rule 105, 106, etc.

At block 520, rule configuration is triggered. For example, the customization manager 330 triggers the rule generator 360 to edit an existing rule, create a new rule, etc. In certain examples, term(s) extracted from the exam can trigger rule creation. For example, a term or set of terms matching a predefine criterion(-ia) can trigger rule editing, rule creation, etc.

At block 525, an existing rule 105, 106 is edited. For example, a rule specifying that a first subset of lab results be displayed in the lab panel 206 and/or be display first in the lab panel 206 can be edited to include a second subset of lab results, exclude a second subset of lab results, replace the first subset with the second subset, move the first subset to be displayed second in the lab panel 206, highlight certain findings/results in the first subset displayed in the panel 206, etc.

At block 530, a new rule 105 is created. For example, a rule can be created by the rule generator 360 to display lab results corresponding to anatomy specified in the reason for exam. A rule can be created to prioritize display of lab results corresponding to a condition involved in the reason for exam, for example. A rule can be created to first display lab results corresponding to a same demographic of the patient, for example. A rule can combine a plurality of terms and/or conditions to display a subset of lab results in the labs panel 206 of the interface 200, for example.

At block 535, the rule 105, 106 is saved. For example, the edited rule is saved in the memory 310 to be used by the processor 101 to generate the layers 111-113 and associated interface 200. At block 550, input captured by the input data processor 320 is evaluated to determine whether additional input is present to drive rule(s) configuration for panel display. If input for rule configuration is available, control reverts to block 520 to configure rule(s) for panel display. If additional input is not available, then, at block 545, an interface 200 definition is generated. For example, a data structure and/or application defining the interface 200 in the user layer 111, admin layer 112, and data layer 113 is generated based on the rule(s), term(s), and other patient/exam information. A hypertext markup language (HTML) file, an extensible markup language (XML) file, a standard generalized markup language (SGML) file, a JavaScript, other executable code, etc., can be used to implement the interface 200 and store its definition. The definition can define the position of components of the interface 200, the rules 105, 106 driving content and behavior of the interface 200, other functionality for display, interaction, and further processing via the interface 200, etc.

At block 550, the interface 200 definition is output. For example, definition of rules 105, 106 for the panel driver 207, patient history 205, labs 206, etc., is provided by the panel generator 370 from output of the rules generator 360 to drive the user interface 200 output by the output processor 380 of the lab interface processor 103.

At block 555, feedback is facilitated. For example, rule edits, rule creation, user interaction, monitored patterns, rule execution results, primary exam processing, image annotation, lab analysis, etc., can be gathered and processed as feedback to impact operation of the processor 101/102/103, configuration of the rules 105, 106, configuration of the interface 200, etc.

FIG. 6 illustrates a flow diagram of a particular example process 600 of rule generation via the example processor 103 (e.g., an example implementation of the process 500). At block 605, the labs 206 section of the interface 200 is accessed. For example, a user accesses the labs category 206 via the processor 101. At block 610, a lab panel and/or order set type is selected for display. For example, the user selects a lab panel and/or order set type via the panel driver 207. At block 615, selection of the lab panel/order set type triggers the lab interface processor 103. For example, the lab interface processor 103 is triggered for rule modification/creation, etc.

At block 620, if the labs are being accessed for the first time (e.g., a first time user login, configuration/setup execution, first time facility access, etc.), then a role (e.g., radiologist, cardiologist, neurologist, pathologist, technician, nurse, surgeon, etc.) and/or subspecialty (e.g., abdominal imaging, breast imaging, cardiac imaging, emergency radiology, musculoskeletal imaging, neurointerventional radiology, neuroradiology, nuclear medicine, pediatric radiology, thoracic imaging, vascular interventional radiology, etc.) is identified. For example, a configuration file, record, profile, preference, input prompt, etc., is processed to identify a role and/or subspecialty associated with a particular user, group of users, location, session, installation, etc.

At block 625, a default set of lab panels and/or order sets is displayed via the interface 200 based on rules associated with the role/subspecialty. For example, for a thoracic imaging subspecialty, arterial blood gas (ABG) analysis, bronchoscopy cell analysis results, etc., are to be displayed alongside lung and other chest images, CT angiography, etc. For a musculoskeletal imaging subspecialty, erythrocyte sedimentation rate (ESR), creatine kinase level, blood test result(s), etc., are to be displayed with x-rays, CT scans, MRIs, etc., for example. At block 630, the default set can be driven by most frequently used labs based on the role/subspecialty, for example. For example, cardiac imaging can be associated with a profile and/or other data structure with an associated set of lab panels/order sets built from feedback and/or other modeling/observation over time.

At block 635, a selection of lab panel(s)/order set(s) is displayed via the interface 200 based on primary exam characteristics. For example, a patient or group of patients for which the interface 200 has been initiated is/are associated with a primary exam record of images, etc., obtained from the patient(s). The primary exam has certain criteria or characteristics associated with it such as through header information (e.g., DICOM header, etc.), annotations, supporting documentation, metadata, etc. Processing of the primary exam can render terms based on these criteria/characteristics. For example, at block 640, data from the primary exam can be provided as display criteria for selection of additional lab panel(s)/order set(s). Primary exam terms/data can include a reason for exam (RFE), an anatomy (e.g., hand, femur, retina, lungs, heart, skeleton, brain, etc.), a condition (e.g., pain, swelling, cancer, deep vein thrombosis (DVT), etc.), a demographic (e.g., smoker, 49 years old, male, married, Polish, etc.), etc. In certain examples, the processor 103 provides natural language processing (NLP) to process the exam record to identify term and recognize associated acronyms, similar terms, etc. Machine learning can be used to combine identified terms and correlate the terms with an associated selection of lab panel(s) and/or order set(s), for example. For example, primary exam terms lung and thoracic can be correlated by a machine learning network model to produce a suggestion of arterial blood gas lab panel, bronchoscopy order set, etc.

At block 645, displayed criteria(-ion) can be confirmed or denied to trigger whether or not to save a rule or rules associated with the displayed criteria(-ion). If, at block 650, a rule is not to be created, then, at block 655, a record of activity is not preserved for the rule and associated criteria(-ion). If, at block 660, a rule is to be created, then, at block 665, rule creation is confirmed. For example, at block 670, rule set(s) can be stored in a preferences panel, and activities and rules can be synchronized to admin 112 and data 113 layers.

At block 675, rule(s) can be viewed, edited, created, etc. For example, one or more rules can be viewed and/or edited, and new rule(s) can be created via the interface 200 and/or another settings/configuration graphical user interface. Rule(s) can be edited/created 665 to impact the displayed lab panel/order set information, modify other rule(s), etc., and/or be generated in addition to rule(s) created 665 in the process above.

At block 680, created rules are displayed via the interface 200, and editable rules can be visually distinguished from uneditable rules in a setting section such as the navigator 202, list 203, panel driver 207, preferences 208, etc. For example, editable user-generated rule sets 105 can be visually distinguished from uneditable system-generated rule sets 106 via the interface 200 and/or other settings/configuration display.

Thus, using the example process 500 and/or 600, driven by the workflow manager 100 and its processors 101-102, rule generation (e.g., rule viewing, rule editing, rule creation, rule execution, etc.) for user interface 200 definition can be triggered (e.g., blocks 510, 615) based on an identified role (e.g., blocks 505, 630) and a primary exam for a patient. Terms can be extracted (e.g., blocks 515, 640) from a record of the primary exam, and the extracted terms and identified role can be processing according to a first set of rules to generate a first lab panel display 206 in the interface 200 (e.g., block 625). Then, rule editing and/or rule generation can be facilitated to form a second set of rules using the extracted terms and the identified role (e.g., blocks 520-540, 635-680). The second set of rules can be an updated first set of rules and/or a separate set of rules in addition to or in place of the first set of rules, for example. The second set of rules is applied to modify the first lab panel display and/or to generate a second lab panel display in addition to or in place of the first lab panel display, for example. An interface definition is generated (e.g., block 545) based on the first lab panel display and the second lab panel display, and the interface definition is provided as output for display and interaction (e.g., block 550). Feedback (e.g., block 555) from usage of the interface 200, modification of the interface 200, etc., can be provided back to drive panel/order set displays (e.g., blocks 625, 635), rules configuration (e.g., blocks 520-540, 645-680), etc.

FIG. 7 is a block diagram of an example processor platform 700 structured to execute the instructions of FIGS. 5-6 to implement the apparatus of FIGS. 1-4. The processor platform 700 can be, for example, a server, a personal computer, a workstation, a self-learning machine (e.g., a neural network), a mobile device (e.g., a cell phone, a smart phone, a tablet such as an iPad′), a personal digital assistant (PDA), an Internet appliance, a DVD player, a CD player, a digital video recorder, a Blu-ray player, a gaming console, a personal video recorder, a set top box, a headset or other wearable device, or any other type of computing device.

The processor platform 700 of the illustrated example includes a processor 712. The processor 712 of the illustrated example is hardware. For example, the processor 712 can be implemented by one or more integrated circuits, logic circuits, microprocessors, GPUs, DSPs, or controllers from any desired family or manufacturer. The hardware processor may be a semiconductor based (e.g., silicon based) device. In this example, the processor 712 implements the example lab interface processor 103.

The processor 712 of the illustrated example includes a local memory 713 (e.g., a cache). The processor 712 of the illustrated example is in communication with a main memory including a volatile memory 714 and a non-volatile memory 716 via a bus 718. The volatile memory 714 may be implemented by Synchronous Dynamic Random Access Memory (SDRAM), Dynamic Random Access Memory (DRAM), RAMBUS® Dynamic Random Access Memory (RDRAM®) and/or any other type of random access memory device. The non-volatile memory 716 may be implemented by flash memory and/or any other desired type of memory device. Access to the main memory 714, 716 is controlled by a memory controller.

The processor platform 700 of the illustrated example also includes an interface circuit 720. The interface circuit 720 may be implemented by any type of interface standard, such as an Ethernet interface, a universal serial bus (USB), a Bluetooth® interface, a near field communication (NFC) interface, and/or a PCI express interface.

In the illustrated example, one or more input devices 722 are connected to the interface circuit 720. The input device(s) 722 permit(s) a user to enter data and/or commands into the processor 712. The input device(s) can be implemented by, for example, an audio sensor, a microphone, a camera (still or video), a keyboard, a button, a mouse, a touchscreen, a track-pad, a trackball, isopoint and/or a voice recognition system.

One or more output devices 724 are also connected to the interface circuit 720 of the illustrated example. The output devices 724 can be implemented, for example, by display devices (e.g., a light emitting diode (LED), an organic light emitting diode (OLED), a liquid crystal display (LCD), a cathode ray tube display (CRT), an in-place switching (IPS) display, a touchscreen, etc.), a tactile output device, a printer and/or speaker. The interface circuit 720 of the illustrated example, thus, typically includes a graphics driver card, a graphics driver chip and/or a graphics driver processor.

The interface circuit 720 of the illustrated example also includes a communication device such as a transmitter, a receiver, a transceiver, a modem, a residential gateway, a wireless access point, and/or a network interface to facilitate exchange of data with external machines (e.g., computing devices of any kind) via a network 726. The communication can be via, for example, an Ethernet connection, a digital subscriber line (DSL) connection, a telephone line connection, a coaxial cable system, a satellite system, a line-of-site wireless system, a cellular telephone system, etc.

The processor platform 700 of the illustrated example also includes one or more mass storage devices 728 for storing software and/or data. Examples of such mass storage devices 728 include floppy disk drives, hard drive disks, compact disk drives, Blu-ray disk drives, redundant array of independent disks (RAID) systems, and digital versatile disk (DVD) drives.

The machine executable instructions 732 of FIGS. 5-6 may be stored in the mass storage device 728, in the volatile memory 714, in the non-volatile memory 716, and/or on a removable non-transitory computer readable storage medium such as a CD or DVD.

FIG. 8 illustrates an alternative implementation of the example processor platform 700. In this example, the processor 712 implements the example patient data processor 101, interface processor 102, and lab interface processor 103.

From the foregoing, it will be appreciated that example methods, apparatus and articles of manufacture have been disclosed that generate an interface and drive panel processing and associated rules according to a primary exam and role/subspecialty associated with an imaging review. The disclosed methods, apparatus and articles of manufacture improve the efficiency of using a computing device by driving tiered, interactive interface definition through automated exam processing, system-generated rules, user-generated rules, and interactive transformative panel displays. The disclosed methods, apparatus and articles of manufacture are accordingly directed to one or more improvement(s) in the functioning of a computer. The disclosed methods, apparatus, and articles of manufacture do not represent mental steps and cannot be performed by a human or in the human mind. Instead, the disclosed methods, apparatus, and articles of manufacture provide interface definition to drive interface display and interaction based on analysis of a primary exam, role/specialty, and both system- and user-generated rules.

Although certain example methods, apparatus and articles of manufacture have been disclosed herein, the scope of coverage of this patent is not limited thereto. On the contrary, this patent covers all methods, apparatus and articles of manufacture fairly falling within the scope of the claims of this patent.

What is claimed is:

1. An apparatus comprising:
memory storing instructions; and
at least one processor to execute the instructions to at least:
trigger rule generation based on an identified role and a primary exam;
extract terms from a record of the primary exam;
process the extracted terms and the identified role according to a first set of rules to generate a first lab panel display in an interface;
facilitate at least one of i) rule editing or ii) rule generation to form a second set of rules using the extracted terms and the identified role;
apply the second set of rules to at least one of:
i) modify the first lab panel display; or
ii) generate a second lab panel display;
generate an interface definition based on the first lab panel display and the second lab panel display; and
provide the interface definition as output for display and interaction.

2. The apparatus of claim 1, wherein the at least one processor includes a lab interface processor.

3. The apparatus of claim 2, wherein the at least one processor further includes a patient data processor and an interface processor.

4. The apparatus of claim 1, wherein the at least one processor is to generate a user layer, a data layer, and an administrative layer to manage the first set of rules and the second set of rules including rule editing and rule generation.

5. The apparatus of claim 1, wherein the first set of rules includes system-generated rules, and wherein the second set of rules includes user-generated rules.

6. The apparatus of claim 1, wherein the at least one processor is further to, based on the interface definition, generate a user interface and arrange the user interface including a panel driver including at least one of the first lab panel display and the second lab panel display positioned and displayed with a primary exam packet, relevant patient history, list items, and a navigator.

7. The apparatus of claim 1, wherein the at least one processor is to process a problem list to extract additional terms.

8. The apparatus of claim 1, wherein the at least one processor is to prompt a user with a selection of lab panel types for display, a frequency of prompting to decrease as user interaction with the selection increases.

9. At least one tangible computer-readable storage medium including instructions that, when executed cause at least one processor to at least:
trigger rule generation based on an identified role and a primary exam;
extract terms from a record of the primary exam;
process the extracted terms and the identified role according to a first set of rules to generate a first lab panel display in an interface;
facilitate at least one of i) rule editing or ii) rule generation to form a second set of rules using the extracted terms and the identified role;
apply the second set of rules to at least one of:
i) modify the first lab panel display; or
ii) generate a second lab panel display;
generate an interface definition based on the first lab panel display and the second lab panel display; and
provide the interface definition as output for display and interaction.

10. The at least one computer-readable storage medium of claim 9, wherein the at least one processor includes a lab interface processor.

11. The at least one computer-readable storage medium of claim 10, wherein the at least one processor further includes a patient data processor and an interface processor.

12. The at least one computer-readable storage medium of claim 9, wherein the instructions, when executed, cause the at least one processor to generate a user layer, a data layer, and an administrative layer to manage the first set of rules and the second set of rules including rule editing and rule generation.

13. The at least one computer-readable storage medium of claim 9, wherein the first set of rules includes system-generated rules, and wherein the second set of rules includes user-generated rules.

14. The at least one computer-readable storage medium of claim 9, wherein the instructions, when executed cause the at least one processor to, based on the interface definition, generate a user interface and arrange the user interface including a panel driver including at least one of the first lab panel display and the second lab panel display positioned and displayed with a primary exam packet, relevant patient history, list items, and a navigator.

15. The at least one computer-readable storage medium of claim 9, wherein the instructions, when executed, cause the at least one processor to process a problem list to extract additional terms.

16. The at least one computer-readable storage medium of claim 9, wherein the instructions, when executed, cause the at least one processor to prompt a user with a selection of lab panel types for display, a frequency of prompting to decrease as user interaction with the selection increases.

17. A method comprising:
triggering, by executing an instruction using at least one processor, rule generation based on an identified role and a primary exam;
extracting, by executing an instruction using the at least one processor, terms from a record of the primary exam;
processing, by executing an instruction using the at least one processor, the extracted terms and the identified role according to a first set of rules to generate a first lab panel display in an interface;
facilitating, by executing an instruction using the at least one processor, at least one of i) rule editing or ii) rule generation to form a second set of rules using the extracted terms and the identified role;
applying, by executing an instruction using the at least one processor, the second set of rules to at least one of:
i) modify the first lab panel display; or
ii) generate a second lab panel display;
generating, by executing an instruction using the at least one processor, an interface definition based on the first lab panel display and the second lab panel display; and
providing, by executing an instruction using the at least one processor, the interface definition as output for display and interaction.

18. The method of claim 17, further including generating a user layer, a data layer, and an administrative layer to manage the first set of rules and the second set of rules including rule editing and rule generation.

19. The method of claim 17, further including, based on the interface definition, generating a user interface and arranging the user interface including a panel driver including at least one of the first lab panel display and the second lab panel display positioned and displayed with a primary exam packet, relevant patient history, list items, and a navigator.

20. The method of claim 17, further including prompting a user with a selection of lab panel types for display, a frequency of prompting to decrease as user interaction with the selection increases.
